(19) United States
(12) Patent Application Publication
(10) Pub. No.: US 2024/0009110 A1
(43) Pub. Date: Jan. 11, 2024

(54) ASSOCIATION OF A VICHY THERMAL WATER AND AN EXTRACT OF AT LEAST ONE BACTERIUM FROM THE SPECIES VITREOSCILLA FILIFORMIS CULTURED ON A MEDIUM COMPRISING AT LEAST ONE VICHY THERMAL WATER

(71) Applicant: L'OREAL, Paris (FR)

(72) Inventors: Audrey GUENICHE, Aulnay-Sous-Bois (FR); Audrey VALOIS, Chevilly La Rue (FR); Lucie GUERIN, Chevilly La Rue (FR)

(21) Appl. No.: 18/251,414

(22) PCT Filed: Nov. 5, 2021
(86) PCT No.: PCT/EP2021/080781

§ 371 Date: May 2, 2023

(30) Foreign Application Priority Data
Nov. 5, 2020 (FR) 2011387

(51) Int. Cl. A61K 8/96; A61Q 19/08; A61K 8/99

(52) U.S. Cl. CPC A61K 8/965; A61Q 19/08; A61K 8/99; A61K 2800/85

(57) ABSTRACT

The present invention relates to an association of a Vichy thermal water and an extract of at least one bacterium from the species Vitreoscilla filiformis cultured on a medium comprising at least one Vichy thermal water, to a composition comprising said association, to a cosmetic treatment process comprising the application to keratin materials of said cosmetic composition for caring for keratin materials, in particular the skin; and also to the cosmetic use of said association for caring for keratin materials, in particular the skin.

The present invention relates to the field of caring for and/or treating keratin materials, in particular the skin.

In particular the present invention relates to an association of a Vichy thermal water and an extract of at least one bacterium from the species Vitreoscilla filiformis cultured on a medium comprising at least one Vichy thermal water, to a composition comprising said association, to a cosmetic treatment process comprising the application to keratin materials of said cosmetic composition for caring for keratin materials, in particular the skin; and also to the cosmetic use of said association for caring for keratin materials, in particular the skin.

Even more particularly, an association of the invention is dedicated to rebalancing cosmetic skin defects, or skin disorders, which are connected with a lack of homeostasis of the innate immune defense system of the skin including endogenous antimicrobial defenses.

The skin constitutes one of the most important organs of the body and is known as being one of the main active elements of the innate immune defense system. Various cell types participate in this system: keratinocytes, melanocytes, certain resident lymphocytes (γδT lymphocytes), and leukocytes such as granulocytes, lymphocytes and macrophages. Among the granulocytes, mention may in particular be made of neutrophil granulocytes also referred to as polynuclear neutrophils, these cells are naturally present in the reticular dermis and are recruited in greater quantity in the skin for example during an attack by an exogenous agent, they participate in particular in the phagocytosis of pathogenic agents and also in the secretion of laminin 5β-3 involved in keratinocyte adhesion as mentioned in The dynamics of the skin's immune system, Alan V. Nguyen and Athena M. Soulika, International Journal of Molecular Sciences, published on 12 Apr. 2019. Thus, these cells play an essential role in the innate immune response and in particular in reepithelialization phenomena and in particular healing phenomena.

Healthy skin is capable of defending itself against various external attacks owing, in particular, to its barrier and antimicrobial defense properties, and also its reepithelialization properties. Nevertheless, continuous attack by the environment, chemicals or radiation may lead to its capacity for adaptation and response, in particular in terms of the innate immune system, being gradually weakened. In the long term, these various attacks may result in a depressant effect on the innate immune defense system, leading to a reduction in the barrier properties and defenses of the skin. These various attacks may also affect the reepithelialization properties and impair the processes of epidermal renewal and healing.

Moreover, the innate immune defense system of the skin tends to become weaker in the course of chronobiological or photoinduced aging. These failures or impairments may be promoted, or even aggravated, by infections due to microorganisms and by the various abovementioned environmental attacks, such as stress, ultraviolet radiation, or “urban” living conditions.

Various damage, resulting from a microbial infection, exposure to UV radiation, or repeated or intense chemical/mechanical attacks, may impair the cells of the epidermis and the operation thereof. This damage is normally controlled by the innate immune defense system which destroys the modified cells. However, during the aging or impairment of the innate immune defense system following environmental attacks, this damage is not correctly taken care of and has the effect of leading to the formation of autoantibodies directed against this damage participating in the establishment of a dysfunction of the skin.

Likewise, the presence of damaged epidermal cells may lead to the reepithelialization and epidermal renewal properties being substantially affected, leading in particular to healing disorders.

Thus, there is a need for new active agents that are useful for caring for keratin materials, in particular capable of promoting homeostasis of the innate immune defense system of the skin.

There is still a need for new active agents for preventing and/or treating cosmetic defects or disorders of the skin that are capable of resulting from a lack of homeostasis of the innate immune defense system of the skin.

There is still a need for new active agents that make it possible to stimulate the defense properties of the skin, and in particular the antimicrobial defense properties.

There is still a need for new useful active agents that are capable of reinforcing the barrier properties of the skin.

There is still a need for new active agents that are useful for reinforcing the reepithelialization, renewal and healing properties of the epidermis.

There is still a need for new active agents for preventing and/or treating the signs of skin aging.

Unexpectedly, the inventors have observed that the exposure of a cellular model of polynuclear neutrophils to an association of Vichy thermal water and an extract of at least one bacterium from the species Vitreoscilla filiformis cultured on a medium comprising at least one Vichy thermal water, makes it possible to stimulate the phagocytosis of particles, via these polynuclear neutrophils, synergistically, thus making it possible to stimulate the immune defense properties of the skin.

To the knowledge of the inventors, no document discloses the association of a Vichy thermal water and an extract of at least one bacterium from the species Vitreoscilla filiformis cultured on a medium comprising at least one Vichy thermal water, and even less the use thereof for stimulating the defense properties of the skin synergistically.

The association of these active agents therefore represents an effective tool both in terms of strengthening the innate immune defense system of the skin and in terms of preventing, reducing, or even slowing down the occurrence of autoimmune disorders, in particular resulting from the aging of the keratin materials or from the attack thereof by various environmental factors, and also in terms of improving reepithelialization phenomena. The strengthening of the innate immune defense system of the skin also makes it possible to reduce, or even to slow down or prevent, the accumulation of damaged epidermal cells, the presence of which leads to the reepithelialization and healing processes of the skin being affected.

The combined effects of each of the active agents of this association has enabled the inventors to define a new active composition, the properties of which prove to be particularly advantageous for caring for keratin materials, notably regarding reepithelialization skin disorders, notably healing disorders, or disorders involving an age-related reepithelialization process, in keratin materials, in particular in the skin.

An association in accordance with the invention advantageously makes it possible to reduce, slow down, prevent or treat cosmetic defects of the skin or skin disorders involving an imbalance of the innate immune defense system, and also to promote the phenomena of reepithelialization or epidermal renewal, and in particular the healing processes.

An association in accordance with the invention is therefore advantageously suitable for caring for keratin materials.

Thus, according to one of its first subjects, the present invention relates to an association comprising a Vichy thermal water and an extract of at least one bacterium from the species Vitreoscilla filiformis cultured on a medium comprising at least one Vichy thermal water.

The present invention also relates to a composition, in particular a cosmetic composition, comprising, in a physiologically acceptable medium, the association according to the invention.

Another of its subjects is a cosmetic treatment process comprising the application to keratin materials, in particular the skin, of the composition according to the invention for caring for keratin materials, in particular the skin, and particularly:
for preventing and/or treating cosmetic skin defects linked to a lack of homeostasis of the innate immune defense system of the skin;
preventing and/or treating cutaneous signs of an external attack, in particular chemical, mechanical or infectious attack, of environmental factors such as variations in climate or exposure to ultraviolet radiation or to tobacco, and/or of exposure to xenobiotics, and/or regulating and/or stimulating the barrier properties of the skin, and/or improving and/or reestablishing the antimicrobial defense properties of the skin, and/or preventing and/or treating the signs of skin aging and/or the signs of skin stress;
preventing and/or treating reepithelialization disorders such as for example a healing disorder;
promoting healing.

Another subject of the present invention is a cosmetic use of the association according to the invention for caring for keratin materials, in particular the skin, and particularly:
for preventing and/or treating cosmetic skin defects linked to a lack of homeostasis of the innate immune defense system of the skin;
preventing and/or treating cutaneous signs of an external attack, in particular chemical, mechanical or infectious attack, of environmental factors such as variations in climate or exposure to ultraviolet radiation or to tobacco, and/or of exposure to xenobiotics, and/or regulating and/or stimulating the barrier properties of the skin, and/or improving and/or reestablishing the antimicrobial defense properties of the skin, and/or preventing and/or treating the signs of skin aging and/or the signs of skin stress;
preventing and/or treating reepithelialization disorders such as for example a healing disorder;
promoting healing.

According to one embodiment, an association in accordance with the invention may be administered topically.

### Definitions

Within the meaning of the invention, the term “keratin materials” aims to denote in particular the skin.

The term “skin” means all of the skin of the body, and the scalp, the skin of the face, neckline and neck, or more preferably still the skin of the face (in particular of the forehead, nose, cheeks, lips and chin).

The term “physiologically acceptable medium” means a medium that is compatible with the skin. According to one particular embodiment, the pH of the cosmetic composition is between 4 and 7.5, notably between 4.5 and 7, and in particular between 4.7 and 6.5.

According to the invention, the term “preventing” or “prevention” means reducing the risk of occurrence or slowing down the occurrence of a given phenomenon.

The term “treating” or “treatment” means any action that aims to improve the comfort or the well-being of an individual; this term thus equally covers attenuating, relieving and curing.

The term “administered topically” means application to the surface of the skin under consideration.

### DETAILED DESCRIPTION

### Extract of at Least One Bacterium from the Species Vitreoscilla filiformis

The extract of at least one bacterium from the species Vitreoscilla filiformis used according to the invention is prepared according to a process comprising the culturing of at least one bacterium from the species Vitreoscilla filiformis on a medium comprising at least one Vichy thermal water.

The species Vitreoscilla filiformis belongs to the Neisseriales order, and more particularly to the Vitreoscilla genus.

These bacteria, several of which have already been described, generally have an aquatic habitat, and may be found especially in marine waters or in thermal waters.

Preferably, the bacterium used within the context of the present invention is that corresponding to the strain deposited at the ATCC under the number 15551.

The term “thermal water” means a hot or cold water which is used for its therapeutic virtues or for spa use. It contains, inter alia, dissolved minerals and trace elements. thermal water is known to be employed for specific treatment purposes depending on the particular trace elements and minerals that it contains.

Within the meaning of the present invention, the term “Vichy thermal water” means thermal water from the Vichy basins.

The Vichy thermal water used in the context of the present invention may have a mineralization of greater than 400 mg/l, in particular greater than 700 mg/l.

In the invention, the term “mineralization” means the sum of the concentrations of anions and cations present in the thermal water.

Moreover, it may have a concentration of bicarbonates of at least 2000 mg/l, and more preferentially of at least 4000 mg/l.

Also, it may have a concentration of silicon oxide of at least 20 mg/l and more preferentially of at least 30 mg/l.

It may also have a concentration of calcium ions of greater than or equal to 50 mg/l, or even 60 mg/l.

The Vichy thermal water comprises in particular from 80 to 120 mg/l of potassium, from 1600 to 2000 mg/l of sodium, from 50 to 200 mg/l of calcium, from 150 to 200 mg/l of sulfates, from 4000 to 5000 mg/l of bicarbonates, from 30 to 70 mg/l of silicon oxides.

Among the waters of the Vichy basin, mention may be made of Lucas water, l′Hôpital water, and Grande Grille water.

In one preferred embodiment, the Vichy water used to culture at least one bacterium of the species Vitreoscilla filiformis is Lucas water.

Culturing may in particular be performed in the following medium:

| Composition | Concentration |
|---|---|
| Autolyzed yeast extract | 0.5 to 5 g/l, preferably 2 to 3 g/l |
| Vegetable peptone | 0.5 to 5 g/l, preferably 2 to 3 g/l |
| Anhydrous glucose | 0.5 to 7 g/l, preferably 2 to 3 g/l |
| Heller microelements | 0.01 to 5 ml/l, preferably 0.1 to 0.3 ml/l |
| CaCl₂•2H₂O | 0.010 to 0.200 g/l, preferably 0.030 to 0.060 g/l |

The volume is made up to 1000 ml with mineral water and/or thermal water optionally supplemented with distilled or osmosed water.

Among the peptones that may be used, an example that may be mentioned is soybean papain peptone.

This medium differs from the media generally used by the absence of catalase and of sulfide.

Heller microelements have been described by Heller, Ann Sci. Nat. Biol. Veg. 14: 1-223 (1953). They are mixtures of various mineral elements which were recommended by Heller, not for culturing bacteria, but for the nutrition of plant tissues cultured in vitro.

The culturing may be carried out at a temperature between 18 and 30° C., preferably between 25 and 27° C. The pH of the culture medium is preferably between 5.5 and 8, for example, the pH of the culture medium is 7.

The composition of the Heller microelements, per 1 L of water, is as follows:

| | | |
|---|---|---|
| ZnSO₄•7H₂O | 1 | g |
| MnSO₄•H₂O | 0.076 | g |
| CuSO₄•5H₂O | 0.003 | g |
| KI | 0.010 | g |
| AlCl₃•6H₂O | 0.050 | g |

The Vichy thermal water may represent all or part of the aqueous phase of the culture medium. It may thus be found as a mixture in any proportion with the water, in particular distilled or osmosed water, present in the culture medium.

Preferably, the mixture (i) of osmosed or distilled water and (ii) of thermal water may be in a (i)/(ii) ratio of between 1 and 100, especially from 90 to 100, in particular from 90 to 99.

After mixing all the elements of the medium, sterilization of the culture medium containing the thermal water is advantageously performed; this step is performed via methods known to those skilled in the art, such as sterilization by filtration or by heat.

The culture medium is then seeded with the bacteria.

The media that are the most suitable for culturing the bacteria are such that the thermal water preferably represents at least 0.1% of the amount of water introduced for the preparation of the medium, especially from 0.1% to 99.9%.

More preferentially, the concentration of thermal water in the medium for culturing the bacteria is between 0.1% and 10%, better still between 0.5% and 5%, more preferably still between 1% and 2%, in particular around 1.3% relative to the osmosed and/or distilled water.

In a known manner, the process for preparing the bacterial extract comprises at least one step in which the bacteria are recovered at the end of culturing, in particular by separating them from the culture medium.

After culturing the bacteria, the biomass may be isolated via various known methods, for example by filtration, by coagulation with an alcohol (ethanol, isopropanol, isobutanol), by drying on a scraped precoat (starch, diatomaceous earth, etc.) roll, or centrifugation. Preconcentration, for example at 80° C. under reduced pressure, improves this separation.

The biomass may be used live or may be treated via various processes. Rupture of the envelopes may be performed, for example via the action of ultrasound. Extracts may also be prepared using an alcohol such as ethanol or propanol.

Lipopolysaccharide extracts may also be prepared according to the known methods: see, for example, Noris and Ribbons, Methods in Microbiology, Vol. 5B, Academic Press (1971). The method generally used is the well-known method known as the Westphal method (or a related method), which consists in performing the extraction with phenol-water mixtures at 65° C. The extract is then subjected to dialysis to remove the phenol.

The bacterial extract that is useful according to the invention may also result from the use of the following process: (i) at least one bacterium from the species Vitreoscilla filiformis is cultured on a medium comprising a saccharide as main source of carbon and at least one mineral or thermal water, and then (ii) after fermentation, the bacteria are separated from the culture medium, to recover said mass of bacteria.

The bacteria recovered after the fermentation step may especially be subjected to a stabilization and/or extraction treatment. It is the extract of at least one bacterium from the species Vitreoscilla filiformis thus obtained that will generally be used in or for the preparation of compositions, in particular cosmetic compositions. In a manner known per se, the extract may thus be sterilized in particular by filtration or by autoclaving.

The term “extract of at least one bacterium from the species Vitreoscilla filiformis” means not only the culture supernatant of said bacteria, but also the biomass obtained after culturing said bacteria or the extracts of the biomass obtained by treating this biomass.

To prepare the extract according to the invention, said bacteria may be cultured via the process according to the invention, and then separated, for example by filtration, centrifugation, coagulation.

Thus, after culturing, the bacteria are concentrated by centrifugation. The biomass obtained is autoclaved. This biomass may be freeze-dried or spray-dried to constitute what is known as the freeze-dried or spray-dried extract. Any freeze-drying or spray-drying method known to those skilled in the art can be used to prepare this extract.

The supernatant fraction of this biomass may also be filtered in a sterile container to remove the particles in suspension. This supernatant fraction may also be transferred aseptically into a sterile container. According to a particular embodiment of the invention, the supernatant fraction thus obtained is used as cosmetic active agent.

The bacterial extract cultured on a medium enriched in thermal water may also be used in the form of fractions of cell components or in the form of metabolites. The microorganism(s), metabolite(s) or fraction(s) may also be introduced in the form of a freeze-dried or spray-dried powder, a culture supernatant and/or, where appropriate, in a concentrated form.

For certain applications, the live biomass may be used in unmodified form, for example in the form of masks or a poultice to produce an immediate effect.

For the purposes of the invention, the term “metabolite” denotes any substance derived from the metabolism of the microorganisms under consideration according to the invention and endowed with efficacy in treating redness of the skin.

The extract of at least one bacterium from the species Vitreoscilla filiformis according to the invention may be present, in a composition, in a concentration of between 0.001% and 20% by weight of dry matter relative to the total weight of the composition, preferably between 0.01% and 10% by weight, better still between 0.1% and 0.5% by weight of dry matter relative to the total weight of the composition.

In a particular embodiment, the extract of at least one bacterium from the species Vitreoscilla filiformis according to the invention may be present in a concentration of 0.01% and 0.1% by weight of dry matter relative to the total weight of the composition.

### Vichy Thermal Water

The Vichy thermal water used in association with the extract of at least one bacterium from the species Vitreoscilla filiformis may have a mineralization of greater than 400 mg/l, in particular greater than 700 mg/l.

In the invention, the term “mineralization” means the sum of the concentrations of anions and cations present in the thermal water.

Moreover, it may have a concentration of bicarbonates of at least 2000 mg/l, and more preferentially of at least 4000 mg/l.

Also, it may have a concentration of silicon oxide of at least 20 mg/l and more preferentially of at least 30 mg/l.

It may also have a concentration of calcium ions of greater than or equal to 50 mg/l, or even 60 mg/l.

The Vichy thermal water comprises in particular from 80 to 120 mg/l of potassium, from 1600 to 2000 mg/l of sodium, from 50 to 200 mg/l of calcium, from 150 to 200 mg/l of sulfates, from 4000 to 5000 mg/l of bicarbonates, from 30 to 70 mg/l of silicon oxides.

Among the waters of the Vichy basin, mention may be made of Lucas water, l′Hôpital water, and Grande Grille water.

Preferably, the Vichy thermal water used in association with the extract of at least one bacterium from the species Vitreoscilla filiformis, is Grande Grille water.

The Vichy thermal water may be present, in a composition, in a concentration of between 0.1% and 95% by weight relative to the total weight of the composition, preferably between 10% and 90% by weight, better still between 20% and 80% by weight relative to the total weight of the composition.

In a one preferred embodiment, the Vichy thermal water is present in a concentration of between 0.1% and 30% by weight relative to the total weight of the composition, preferably between 1% and 25%, more preferably 10% a 20% by weight relative to the total weight of the composition.

In another preferred embodiment, the Vichy thermal water is present in a concentration of between 60% and 80% by weight relative to the total weight of the composition.

The association of (i) said Vichy thermal water and (ii) said extract of at least one bacterium from the species Vitreoscilla filiformis cultured on a medium comprising at least one Vichy thermal water are present in a (ii)/(i) weight ratio of between 0.0001 and 0.1, preferably between 0.001 and 0.05, better still between 0.002 and 0.03.

### Composition

The present invention also relates to a composition, in particular a cosmetic composition, comprising, in a physiologically acceptable medium, the association of a Vichy thermal water and an extract of at least one bacterium from the species Vitreoscilla filiformis cultured on a medium comprising at least one Vichy thermal water as defined above.

Said extract may be present, in a composition, in a concentration of between 0.001% and 20% by weight of dry matter relative to the total weight of the composition, preferably between 0.01% and 10% by weight, better still between 0.1% and 0.5% by weight of dry matter relative to the total weight of the composition.

In a particular embodiment, said extract may be present, in a composition, in a concentration of 0.01% and 0.1% by weight of dry matter relative to the total weight of the composition.

Said thermal water may be present, in a composition, between 5% and 95% by weight relative to the total weight of the composition, preferably between 10% and 90% by weight, better still between 20% and 80% by weight relative to the total weight of the composition.

The term “physiologically acceptable medium” means a medium that is compatible with keratin materials and in particular the skin.

More particularly, said physiologically acceptable medium may comprise water and/or one or more water-miscible organic solvents which may be chosen from linear or branched C1-C6 monoalcohols such as ethanol, isopropanol or tert-butanol; polyols such as glycerol, propylene glycol, hexylene glycol (or 2-methyl-2,4-pentanediol), and polyethylene glycols; polyol ethers such as dipropylene glycol monomethyl ether; and mixtures thereof.

The composition according to the invention may have a content of water other than the Vichy thermal water, ranging from 5% to 95% by weight, better still from 10% to 90% by weight, better still between 20% and 80% by weight relative to the total weight of the composition.

In one preferred embodiment, the composition according to the invention does not comprise any water other than the Vichy thermal water.

Advantageously, the composition comprises one or more water-miscible organic solvents in a content ranging from 0.5% to 25% by weight, preferably from 1% to 20% by weight, better still from 5% to 10% by weight, relative to the total weight of the composition.

The composition according to the invention may comprise water and/or any adjuvant normally used in the envisaged field of application.

Mention may notably be made of organic solvents, notably C1-06 alcohols and C2-C10 carboxylic acid esters; carbon-based and/or silicone oils, of mineral, animal and/or plant origin; water, waxes, pigments, fillers, colorants, surfactants, emulsifiers, coemulsifiers; cosmetic or dermatological active agents such as vitamin C, UV-screening agents, polymers, hydrophilic or lipophilic gelling agents, thickeners, preserving agents, fragrances, bactericides, odour absorbers and antioxidants.

These optional adjuvants may be present in the composition in a proportion of from to 80% by weight, preferably 0.01% to 40% by weight, better still from 0.1% to 20%, relative to the total weight of the composition. Depending on their nature, these adjuvants may be introduced into the fatty phase, into the aqueous phase and/or into the lipid vesicles.

The compositions according to the invention may be in any presentation form conventionally used for topical application and notably in the form of aqueous or aqueous-alcoholic solutions, oil-in-water (O/W), water-in-oil (W/O) or multiple (triple: W/O/W or emulsions, aqueous gels, or dispersions of a fatty phase in an aqueous phase using spherules, these spherules possibly being lipid vesicles of ionic and/or nonionic type (liposomes, niosomes or oleosomes). These compositions are prepared according to the usual methods.

Advantageously, the compositions according to the invention are in the form of a gel, of an emulsion, of a powder or of a paste. In addition, the composition according to the invention may be more or less fluid and may have the appearance of a white or colored cream, an ointment, a milk, a lotion, a serum, a paste, a foaming gel, a scrub, a mask, a care product, a tonic or a foam. It may optionally be applied to the skin in aerosol form. It may also be in solid form, for example in stick form.

A composition according to the invention may comprise an oily phase.

A composition used according to the invention may advantageously comprise at least one liquid fatty substance.

The term “liquid fatty substance” means a compound with a melting point below about as opposed to solid fatty substances, such as waxes, which have a melting point above about 50° C.

As oils that may be used in the composition of the invention, examples that may be mentioned include:
hydrocarbon-based oils of animal origin;
hydrocarbon-based oils of plant origin,
synthetic esters and ethers, especially of fatty acids, for instance the oils of formulae R′000R2 and R′0R2 in which R′ represents a fatty acid residue containing from 8 to 29 carbon atoms, and R2 represents a branched or unbranched hydrocarbon-based chain containing from 3 to 30 carbon atoms;
linear or branched hydrocarbons of mineral or synthetic origin;
fatty alcohols having from 8 to 26 carbon atoms;
partially hydrocarbon-based and/or silicone-based fluoro oils;
silicone oils;
mixtures thereof.

In the list of oils mentioned above, the term “hydrocarbon-based oil” means any oil mainly including carbon and hydrogen atoms, and possibly ester, ether, fluoro, carboxylic acid and/or alcohol groups. The other fatty substances that may be present in the oily phase are, for example, fatty acids including from 8 to 30 carbon atoms, waxes, silicone resins and silicone elastomers. These fatty substances may be chosen in a varied manner by a person skilled in the art in order to prepare a composition having the desired properties, for example in terms of consistency or texture.

According to a particular embodiment of the invention, the composition according to the invention is a water-in-oil (W/O) or oil-in-water (O/W) emulsion. The proportion of the oily phase of the emulsion may range from 5% to 90% by weight and preferably from 5% to 60% by weight relative to the total weight of the composition. The emulsions generally contain at least one emulsifier chosen from amphoteric, anionic, cationic and nonionic emulsifiers, used alone or as a mixture, and optionally a coemulsifier. The emulsifiers are chosen in an appropriate manner according to the emulsion to be obtained (W/O or O/W emulsion). The emulsifier and the coemulsifier are generally present in the composition in a proportion ranging from 0.3% to 30% by weight and preferably from 0.5% to 20% by weight relative to the total weight of the composition.

For the W/O emulsions, examples of emulsifiers that may be mentioned include dimethicone copolyols and alkyldimethicone copolyols. A crosslinked elastomeric solid organopolysiloxane including at least one oxyalkylene group may also be used as W/O emulsion surfactant.

For the O/W emulsions, examples of emulsifiers that may be mentioned are nonionic emulsifiers.

The association according to the invention, or said composition comprising same, may be used topically in the context of a use or of a process according to the invention.

The compositions according to the invention may be applied directly to the skin or, alternatively, to cosmetic supports of occlusive or non-occlusive type, intended to be applied locally to the skin. As non-limiting examples of cosmetic supports, mention may be made notably of a patch, a wipe, a roll-on and a pen. The composition may optionally be rinsed off after having been applied to the skin.

### Cosmetic Treatment Process and Cosmetic Uses

Another of its subjects is a cosmetic treatment process comprising the application to keratin materials, in particular the skin, of the composition according to the invention for caring for keratin materials, in particular the skin, and particularly:
for preventing and/or treating cosmetic skin defects linked to a lack of homeostasis of the innate immune defense system of the skin;
preventing and/or treating cutaneous signs of an external attack, in particular chemical, mechanical or infectious attack, of environmental factors such as variations in climate or exposure to ultraviolet radiation or to tobacco, and/or of exposure to xenobiotics, and/or regulating and/or stimulating the barrier properties of the skin, and/or improving and/or reestablishing the antimicrobial defense properties of the skin, and/or preventing and/or treating the signs of skin aging and/or the signs of skin stress;
preventing and/or treating reepithelialization disorders such as for example a healing disorder;
promoting healing.

Another subject of the present invention is a cosmetic use of the association according to the invention for caring for keratin materials, in particular the skin, and particularly:
for preventing and/or treating cosmetic skin defects linked to a lack of homeostasis of the innate immune defense system of the skin;
preventing and/or treating cutaneous signs of an external attack, in particular chemical, mechanical or infectious attack, of environmental factors such as variations in climate or exposure to ultraviolet radiation or to tobacco, and/or of exposure to xenobiotics, and/or regulating and/or stimulating the barrier properties of the skin, and/or improving and/or reestablishing the antimicrobial defense properties of the skin, and/or preventing and/or treating the signs of skin aging and/or the signs of skin stress;
preventing and/or treating reepithelialization disorders such as for example a healing disorder;
promoting healing.

The cutaneous or epidermal conditions concerned by the invention fall under epidermises presenting signs resulting from a lack of homeostasis of the innate immune defense system of the skin, including endogenous antimicrobial defenses and/or a lack of reepithelialization processes, and preferably a lack of both these aspects.

More particularly, the cutaneous conditions concerned by the invention may be as described below.

A lack of homeostasis of the innate immune defense system of the skin and/or of reepithelialization processes may result from various phenomena such as exposure to external attacks, in particular chemical, mechanical or infectious attacks, environmental factors, such as variations in climate or exposure to ultraviolet rays or to tobacco, and/or to xenobiotics, such as for example microorganisms.

In particular, a lack of homeostasis of the innate immune defense system of the skin and/or of reepithelialization processes may occur following external attacks of the following types: UV radiation, irritants, such as detergents, acids, bases, oxidizing agents, reducing agents, concentrated solvents, toxic gases or smoke; mechanical stresses, such as friction, impacts, abrasions, tearing of the surface of the skin, the spraying of dust or of particles, shaving or epilation; thermal or climatic imbalances, such as the cold, dryness, or UV radiation; xenobiotics, such as undesirable microorganisms or allergens; or following internal stress attacks of endogenous origin such as disorders involving an inflammatory and/or hormonal mechanism affecting the skin, mucous membrane and/or the scalp. Physiological endogenous stress may, for example, be linked to the abnormal production of proinflammatory mediators such as neuromediators, cytokines or chemokines, giving rise, in particular, to an impairment of the cutaneous barrier function.

Also, a lack of homeostasis of the innate immune defense system of the skin and/or of reepithelialization processes may be caused or aggravated by chronobiological or photoinduced aging of the skin.

According to one embodiment, an association in accordance with the invention may advantageously be used as an active agent useful for preventing and/or treating the skin signs resulting from an external attack, in particular chemical, mechanical or infectious attacks, environmental factors, such as variations in climate or exposure to ultraviolet rays or to tobacco, and/or exposure to xenobiotics.

According to one embodiment, an association in accordance with the invention may advantageously be used as an active agent useful for regulating and/or simulating the barrier properties of the skin.

According to one embodiment, an association in accordance with the invention may advantageously be used as an active agent useful for improving and/or reestablishing the antimicrobial defense properties of the skin.

According to one embodiment, an association in accordance with the invention may advantageously be used as an active agent useful for preventing and/or treating the signs of skin aging.

The term “signs of skin aging” is intended to mean all the modifications of the external appearance of the skin due to aging, whether the latter is of chronological and/or photoinduced origin.

By way of example of these modifications considered in the invention, mention may be made of a surface which is not very uniform and less smooth, a thinned epidermis, wrinkles and fine lines, withered skin, a lack of elasticity and/or of tonicity of the skin, thinning of the dermis and/or the degradation of collagen fibers, which leads to the appearance of flaccid, wrinkled skin.

In particular, the signs of skin aging targeted by the invention are chosen from a thinning of the skin, a loss of firmness, a loss of elasticity, a loss of density or a loss of tonicity of the skin, a deterioration of the surface appearance of the skin, the appearance of a marked microrelief of the skin, the appearance of roughness, the formation and/or presence of fine lines and/or of wrinkles, a deterioration of the radiance of the skin complexion, a papery appearance of the skin, a sagging of the skin, or a withering of the skin.

Preferably, the signs of skin aging targeted by the invention are chosen from a loss of firmness, a loss of elasticity, a loss of tonicity of the skin, a deterioration of the surface appearance of the skin, the appearance of a marked microrelief of the skin, the appearance of roughness, the formation and/or presence of fine lines and/or of wrinkles, a deterioration of the radiance of the skin complexion, a papery appearance of the skin, a withering of the skin.

More preferably, the signs of skin aging targeted by the invention are chosen from a deterioration of the surface appearance of the skin, the appearance of a marked microrelief of the skin, the appearance of roughness, the formation and/or presence of fine lines and/or of wrinkles, a deterioration of the radiance of the skin complexion.

According to another embodiment, an association in accordance with the invention may advantageously be used as an active agent useful for preventing and/or treating the signs of skin stress.

Stress-induced skin signs may in particular be reflected by feelings of skin discomfort, sensory phenomena and/or the manifestation of undesirable or even painful skin signs, such as irritation or a feeling of discomfort of the skin which may be manifested in particular by stinging, tautness, hotness and/or itching. They may also be redness, itching, hotness, burning sensations, stinging sensations, tautness. Other visible skin signs may also appear, such as pruritus, dry patches, inflammatory erythema, edema and/or pimples, or else skin irritation reactions.

According to another embodiment, an association in accordance with the invention may advantageously be used as active agent useful for the preparation of a composition for preventing and/or treating skin disorders linked to an imbalance of homeostasis of the innate immune defense system of the skin.

According to another embodiment, the skin disorder may be chosen from disorders of reepithelialization, in particular healing.

Advantageously, an association in accordance with the invention may advantageously be suitable for promoting and/or enhancing healing phenomena.

According to one embodiment, an association in accordance with the invention may advantageously be used to prevent and/or treat chronic or acute healing defects, in particular associated with chronobiological aging and/or photoinduced aging of the skin.

Throughout the description, including the claims, the term “comprising a” should be understood as being synonymous with “comprising at least one”, unless otherwise specified.

In addition, the term “at least one” should be understood as being synonymous with “one or more”, unless otherwise specified.

The terms “more than”, “between . . . and . . . ” and “ranging from . . . to . . . ” should be understood as being limits inclusive, unless otherwise specified.

The examples and figures that follow are presented as non-limiting illustrations of the invention. The compounds are, depending on the case, cited as the chemical names or as the CTFA (International Cosmetic Ingredient Dictionary and Handbook) names.

### EXAMPLES

### Example 1—Preparation of an Extract of Bacteria According to the Invention: Vitreoscilla filiformis Biomass Cultured on a Medium Enriched with Vichy Thermal Water

Preparation of the Culture Medium:

Composition:

| | | |
|---|---|---|
| Yeast extract | 2 | g |
| Soybean papain peptone | 2 | g |
| Glucose | 2 | g |
| Heller microelement | 0.2 | ml |
| CaCl₂•2H₂O | 50 | mg |
| Vichy thermal water | 10.04 | ml. |

This stock solution will be diluted with osmosed water in a proportion of 1/75 before sterilization.

The medium is sterilized by autoclaving at 121° C. for 20 minutes. After cooling to room temperature, the pH is adjusted to 7.0 by adding a molar NaOH or KOH solution.

Culture

After seeding the medium at 1% with the Vitreoscilla filiformis ATCC 15551 strain, the culture is placed under orbital shaking at 100 rpm and at 26° C. After 48 hours of growth, the culture is centrifuged at 4500×g for 15 minutes. The pellets are recovered and adjusted to between 4.0% and 4.5% by weight of dry matter with the supernatant resulting from the centrifugation, and are then autoclaved at 121° C. for 30 minutes. This biomass may be used for evaluation tests.

### Example 2—Activity of the Bacterial Extract According to the Invention (Vitreoscilla Filiformis Biomass Cultured on a Medium Enriched with Vichy Thermal Water)

Experimental Conditions

Cell type: Granulocytes (polynuclear neutrophils, PMNs) isolated from the peripheral blood stream of 27-year-old male donors using the Ficoll gradient and lysis of the erythrocytes.

Culture conditions: 37° C., 5% CO₂.

Culture Medium
standard culture medium: RPMI-1640 supplemented with 2 mM L-glutamine, 100 U/ml penicillin, 0.1% bovine serum albumin (BSA).
personalized culture medium: Powdered RPMI-1640 (Sigma reference R6504) reconstituted either with ultrapure water, or with Vichy thermal water (Grande Grille spring) filtered at 0.22 μm and supplemented with 100 U/ml penicillin, 100 μg/ml streptomycin and 0.1% bovine serum albumin (BSA).

Culture and Treatment

The PMNs freshly isolated from the peripheral bloodstream are seeded on 96-well plates in the personalized culture medium as defined above containing or not containing (control) the compounds to be tested, combinations thereof, or the reference compound (IL-8 tested at 10 ng/ml). The cells were preincubated for 10 minutes at 37° C. Next, fluorescent particles (Molecular Probes FluoSpheres™ Carboxylates-Modified Microspheres, 1.0 μm, Invitrogen™ ref F8823) were added and the cells were incubated for 45 minutes. A control with cells in the absence of fluorescent particles was performed simultaneously.

At the same time, for validation of the test, an additional control was performed under the standard conditions of Bioalternatives using the standard culture medium as defined above.

All the experimental conditions are performed 3 times, except for the control which was performed 6 times.

After incubation, the cells are rinsed in PBS/0.1% BSA while performing a centrifugation step. Data acquisition was carried out on a total of 10 000 cells for each of the replicates using a BD FACVerse™ flow cytometer. The data is analyzed using the FlowJo v10.5.2 software.

The phagocytosis of the fluorescent particles by the PMNs was estimated by determining the measurement window (i.e. gating) and analysis of the FITC (fluorescein isothiocyanate) positive PMNs within the population of living cells and the results are expressed as % of FITC (fluorescein isothiocyanate) positive PMNs.

The results are given in table 1 below.

TABLE 1

| | | | FITC | | | | | |
|---|---|---|---|---|---|---|---|---|
| | | | positive | | Standard | % | Standard | P |
| Compound tested | | [C] | PMNs | Mean | deviation | control | deviation | value |
| Standard | Control | — | 54.8 | 50.0 | 3.9 | 100 | 8 | — |
| culture | | | 52.9 | | | | | |
| medium | | | 42.2 | | | | | |
| | IL-8 | 10 ng/ml | 72.7 | 67.6 | 3 | 135 | 6 | * |
| | | | 67.9 | | | | | |
| | | | 62.3 | | | | | |
| Person- | Control | — | 50.2 | 45.4 | 2.1 | 100 | 5 | * |
| alized | | | 37.7 | | | | | |
| culture | | | 48.0 | | | | | |
| medium | | | 50.5 | | | | | |
| | | | 41.1 | | | | | |
| | | | 45.1 | | | | | |
| | IL-8 | 10 ng/ml | 76.3 | 72.0 | 3.2 | 158 | 7 | *** |
| | | | 74.0 | | | | | |
| | | | 65.7 | | | | | |
| | Extract | 0.1% | 69.5 | 72.4 | 1.4 | 159 | 3 | *** |
| | obtained | | 74.0 | | | | | |
| | according | | 73.6 | | | | | |
| | to | 0.3% | 54.0 | 58.5 | 1.7 | 124 | 4 | * |
| | example | | 55.9 | | | | | |
| | 1 | | 59.7 | | | | | |
| | Vichy | 16.7% | 39.2 | 45.8 | 3.5 | 101 | 8 | ns |
| | thermal | | 47.1 | | | | | |
| | water | | 51.1 | | | | | |
| | (Grande | 50% | 51.2 | 57.6 | 3.8 | 127 | 8 | * |
| | Grille | | 57.3 | | | | | |
| | spring) | | 64.4 | | | | | |
| | Mixture | 0.1% + | 79.5 | 77.2 | 1.5 | 170 | 3 | *** |
| | of the | 16.7% | 77.6 | | | | | |
| | extract | | 74.4 | | | | | |
| | obtained | 0.3% + | 72.3 | 69.9 | 2.5 | 154 | 5 | *** |
| | according | 50% | 72.4 | | | | | |
| | to | | 64.9 | | | | | |
| | example | | | | | | | |
| | 1 and | | | | | | | |
| | Vichy | | | | | | | |
| | thermal | | | | | | | |
| | water | | | | | | | |
| | (Grande | | | | | | | |
| | Grille | | | | | | | |
| | spring) | | | | | | | |

[C]: weight concentrations
ns > 0.05: not significant.
* 0.01 to 0.05: significant.
** 0.001 to 0.01: very significant.
*** <0.001: very highly significant.

Observations

The extract obtained according to example 1 shows a clear and significant increase in the phagocytosis of the fluorescent particles by the PM Ns at all the concentrations tested (159%, and 124% of the control).

The Vichy thermal water tested at 16.7% and 50% also shows a significant effect of stimulation of the phagocytosis by the PM Ns but at the highest concentration (127% of the control).

The association of the extract obtained according to example 1 and the Vichy thermal water shows a greater effect than the effect of the two compounds tested individually, thus showing a synergy effect of these two compounds in combination. By way of example: the effect of the extract obtained according to example 1 at 0.1% is +59% compared to the control, the effect of the Vichy thermal water, the effect of the Vichy thermal water at 16.7% is +1% compared to the control, whereas the effect of the association of the extract obtained according to example 1 and the Vichy thermal water is +70% compared to the control (+70%>59%+1%).

Conclusion

The association of the extract obtained according to example 1 and the Vichy thermal water therefore exhibits a synergy effect on the phagocytosis of particles by the PM Ns and therefore makes it possible to stimulate the skin immune defenses.

### Example 3—Cosmetic Composition

The composition presented in table 2 below was prepared according to the standard methods of the person skilled in the art.

TABLE 2

| INCI Name | Concentration (g) |
|---|---|
| PEG/PPG/POLYBUTYLENE GLYCOL-8/5/3 | 2 |
| GLYCERIN | |
| HYDROXYACETOPHENONE | 0.50 |
| PROPANEDIOL | 3 |
| METHYL GLUCETH-20 | 1 |
| GLYCERIN | 1.90 |
| BUTYLENE GLYCOL | 0.80 |
| CAPRYLYL GLYCOL | 0.20 |
| VICHY THERMAL WATER (GRANDE | 79.80 |
| GRILLE SPRING) | |
| NIACINAMIDE | 4 |
| CITRIC ACID MONOHYDRATE | 0.35 |
| SODIUM HYALURONATE | 0.40 |
| CARBOMER | 0.35 |
| BIOSACCHARIDE GUM-1 | 0.20 |
| EXTRACT OBTAINED ACCORDING TO | 5 |
| EXAMPLE 1 (4.25% by weight of dry matter) | |
| OXYETHYLENATED (60 EO) | 0.30 |
| HYDROGENATED CASTOR OIL | |
| VITAMIN E | 0.20 |

The composition was then applied to the skin of the face and exhibits properties that stimulate the immune defenses of the skin, and also antiaging properties.

1. An association comprising a Vichy thermal water and an extract of at least one bacterium from the species Vitreoscilla filiformis cultured on a medium comprising at least one Vichy thermal water.

2. The association as claimed in claim 1, wherein (i) said Vichy thermal water and (ii) said extract of at least one bacterium from the species Vitreoscilla filiformis cultured on a medium comprising at least one Vichy thermal water are present in a (ii)/(i) weight ratio of between 0.0001 and 0.1.

3. The association as claimed in claim 1, wherein said bacterium corresponds to the strain deposited at the ATCC under the number 15551.

4. A composition comprising, in a physiologically acceptable medium, the association as defined in claim 1.

5. The composition as claimed in claim 4, wherein said Vichy thermal water is present in a concentration of between 0.1% and 95% by weight relative to the total weight of the composition.

6. The composition as claimed in claim 4, wherein said extract is present in a concentration of between 0.001% and 20% by weight of dry matter relative to the total weight of the composition.

7. A process of caring for keratin materials comprising applying to keratin materials the composition as defined in claim 1.

8. A process of treating cosmetic skin defects linked to a lack of homeostasis of the innate immune defense system of skin comprising applying to keratin materials the composition as defined in claim 1.

9. A process of treating signs of skin aging comprising applying to keratin materials the composition as defined in claim 1.

10. (canceled)

11. (canceled)

12. (canceled)

13. The process as claimed in claim 9, wherein the signs of skin aging are at least one chosen from a thinning of the skin, a loss of firmness, a loss of elasticity, a loss of density, a loss of tonicity of the skin, a deterioration of the surface appearance of the skin, the appearance of a marked microrelief of the skin, the appearance of roughness, the presence of fine lines and/or of wrinkles, a deterioration of the radiance of the skin complexion, a papery appearance of the skin, a sagging of the skin, and a withering of the skin.

14. The association as claimed in claim 1, wherein (i) said Vichy thermal water and (ii) said extract of at least one bacterium from the species Vitreoscilla filiformis cultured on a medium comprising at least one Vichy thermal water are present in a (ii)/(i) weight ratio of between 0.001 and 0.05.

15. The association as claimed in claim 2, wherein said bacterium corresponds to the strain deposited at the ATCC under the number 15551.

16. A composition comprising, in a physiologically acceptable medium, the association as defined in claim 2.

17. The composition as claimed in claim 4, wherein said Vichy thermal water is present in a concentration of between 10% and 90% by weight relative to the total weight of the composition.

18. The composition as claimed in claim 4, wherein said extract is present in a concentration of between 0.01% and 10% by weight of dry matter relative to the total weight of the composition.

19. The process as claimed in claim 9 wherein the signs of skin aging are chosen from the presence of fine lines and/or of wrinkles
